(19) United States
(12) Patent Application Publication
(10) Pub. No.: US 2005/0223369 A1
(43) Pub. Date: Oct. 6, 2005

(54) Method and system for programming a reconfigurable processing element

(75) Inventors: Anthony L. Chun, Los Altos, CA (US); Vicki W. Tsai, Mountain View, CA (US); Walter L. Snyder, San Jose, CA (US); Siva Simanapalli, Santa Clara, CA (US)

Correspondence Address: VENABLE LLP, P.O. BOX 34385, WASHINGTON, DC, 20045-9998, US

(73) Assignee: Intel Corporation, Santa Clara, CA (US)

(21) Appl. No.: 10/813,596

(22) Filed: Mar. 31, 2004

(51) Int. Cl. G06F 9/45; A61K 31/18

(52) U.S. Cl. 717/136

(57) ABSTRACT

A programming tool parses and converts instructions for use in a reconfigurable processing element, such as a filter micro-coded accelerator.

### BACKGROUND OF THE INVENTION

Communications devices are being developed and introduced into an evolving communications landscape that includes an increasing number of network protocols. It may be necessary for the devices to support multiple protocols. To support existing and new protocols, devices may need to be reprogrammed to accept alternative or new protocols.

Assembly language may provide a traditional programming model, but many communications devices do not have traditional processor architectures. Developments may be needed to provide programming tools for these communications devices.

### BRIEF DESCRIPTION OF THE DRAWINGS

The invention shall be described with reference to the accompanying figures, wherein:

FIG. 1 illustrates a diagram of a programming tool, according to an embodiment of the present invention;

FIG. 2 illustrates a diagram of a filter processing element (PE) including a reconfigurable control unit (RCU), according to an embodiment of the present invention;

FIG. 3 illustrates a diagram of a RCU for the PE, according to embodiments of the present invention;

FIG. 4 illustrates a programming output example for the PE, according to embodiments of the present invention;

FIG. 5 illustrates a flowchart of the programming operations, according to an embodiment of the present invention; and

FIG. 6 illustrates a computing environment, which may be implemented in one or more devices, such as, but not limited to communications devices, according to embodiments of the present invention.

The invention is now described with reference to the accompanying drawings. In the drawings, like reference numbers generally indicate identical, functionally similar, and/or structurally similar elements. The drawing in which an element first appears is generally indicated by the left-most digit(s) in the corresponding reference number.

### DETAILED DESCRIPTION OF PREFERRED EMBODIMENTS

One or more micro-coded accelerators (MCA) may be implemented in processing elements (PE). MCAs may be implemented such that they are able to support multiple protocols by the inclusion of control logic that may be reconfigurable. While the present invention may be described in terms of the embodiments provided herein, this is exemplary and is not intended to limit its application. In fact, after reading the following description, it will be apparent to one of ordinary skill in the art(s) how to implement the following invention in alternative embodiments (e.g., in various communications devices and operating the programming tool and the operations of the programming tool with various MCAs with various protocols).

Furthermore, while the following description focuses on programming from assembly language, it is not intended to limit the application of the present invention to the current scope of programming languages. It will be apparent to one skilled in the relevant art(s) how to implement the following invention, where appropriate, in alternative embodiments. For example, the present invention may be applied, alone or in combination, in various PE architectures, to convert, combine and deliver programming from object-oriented programming languages.

In this detailed description, numerous specific details are set forth. However, it should be understood that embodiments of the invention may be practiced without these specific details. In other instances, well-known circuits, structures, and/or techniques have not been shown in full detail to aid an understanding of the present invention.

References to “one embodiment”, “an embodiment”, “example embodiment”, “various embodiments”, etc., indicate that the embodiment(s) of the invention so described may include a particular feature, structure, or characteristic, but not every embodiment necessarily includes the particular feature, structure, or characteristic. Further, repeated use of the phrase “in one embodiment” does not necessarily refer to the same embodiment, although it may.

In this detailed description and claims, the term “coupled,” along with its derivatives, such as, “connected” and “electrically connected”, may be used. It should be understood that “coupled” may mean that two or more elements are in direct physical or electrical contact with each other or that the two or more elements are not in direct contact but still cooperate or interact with each other.

An algorithm may be here, and generally, considered to be a self-consistent sequence of acts or operations leading to a desired result. These include physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers or the like. It should be understood, however, that all of these and similar terms are to be associated with the appropriate physical quantities and are merely convenient labels applied to these quantities.

Unless specifically stated otherwise, as apparent from the following discussions, it is appreciated that throughout the specification discussions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

In a similar manner, the term “processor” may refer to any device or portion of a device that processes electronic data from registers and/or memory to transform that electronic data into other electronic data that may be stored in registers and/or memory. As such, a processor may not be a fully implemented central processing unit, but rather a specialized element configured to perform one or more specific tasks. A “computing platform” may comprise one or more processors. The one or more processors may be implemented within the same processor core.

Embodiments of the present invention may include apparatuses for performing the operations herein. An apparatus may be specially constructed for the desired purposes, or it may comprise a general purpose device selectively activated or reconfigured by a program stored in the device.

Embodiments of the invention may be implemented in one or a combination of hardware, firmware, and software. Embodiments of the invention may also be implemented as instructions stored on a machine-readable medium, which may be read and executed by a computing platform to perform the operations described herein. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other form of propagated signals (e.g., carrier waves, infrared signals, digital signals, etc.), and others.

The present invention may provide an automated programming tool for converting assembly code into the proper configuration to be delivered by download to PE that performs filtering, decoding, encoding, and other signal processing operations as one of ordinary skill in the art would recognize based at least on the teachings provided herein. In one embodiment, a filter MCA (FMCA) may be reconfigured, but the programming method, system and tool of the present invention is not limited to an FMCA. A FMCA may perform, according to some embodiments, computationally intensive physical (PHY) layer processing, such as, but not limited to, filtering, resampling, FFT, channel estimation, dispreading, and error correction decoding.

The FMCA may be implemented within a reconfigurable programmable architecture. In embodiments, a reconfigurable FMCA may include a control unit (CU) with a non-traditional instruction memory, such as, but not limited to a reconfigurable programmable logic array (RPLA). In embodiments of the present invention, the RPLA may generate control signals for the FMCA's datapath.

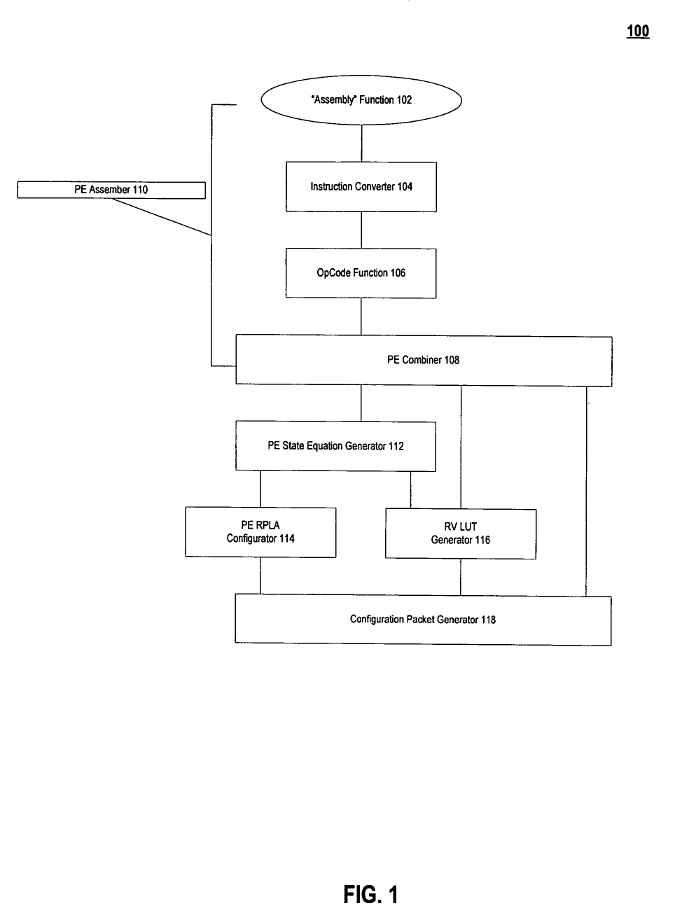

With respect to FIG. 1, a diagram of a programming tool 100, according to an embodiment of the present invention, is shown. Tool 100 may include a processing element (PE) assembler 110. The assemble 110 may include, according to embodiments of the present invention, assembly function 102, instruction converter 104, operation code function 106, and PE combiner 108.

According to embodiments of the present invention, the instruction converter 104 may receive a function in assembly code and parse the function into a parsed group selected from a group including an instruction, data, a constant, a coefficient, or a factor. Once the function may be parsed, the instruction converter 104 may convert one or more of the parsed group into one or more operation codes. An operation code may be a numeric representation for each of the parsed group.

A processing element combiner 108 may determine the one or more operation codes to be executed on a processing element and combine the one or more operation codes into a file. The combiner 108 may be coupled to a state equation generator 112, a reconfigurator vector (RV) look-up table generator 116, and a configuration packet generator 118.

The state equation generator 112 may convert the file into a state machine representation. The instruction converter 112 may assign an operation number as an output of a state of the one or more operation codes, determine a next state for each of the one or more operation codes, wherein the next state may be a function of the state and an input signal, associate the operation number with a reconfigurator vector, and generate a state equation for each state.

According to embodiments of the present invention, a reconfigurable logic array configurator 114 may map the state equations to a control unit reconfigurable logic array, and program a fuse map for the control unit reconfigurable logic array. The configurator 114 may forward, at least the fuse map to a configuration packet generator 118.

The reconfigurator vector look-up table 116 may produce an output reconfigurator vector look-up table, where the reconfigurator vector look-up table converts the operation number to a reconfigurator vector. According to embodiments of the present invention, at least the reconfigurator vector may be forwarded to the configuration packet generator 118.

In embodiments of the present invention, the configuration packet generator 118 may create a configuration packet used to program the processing element. The configuration packet may include at least one of the fuse map, the output reconfigurator vector look-up table, the parsed group, one or more fuse maps for other control unit reconfigurable logic arrays, routing information, one or more processing element addresses, or packet sizes.

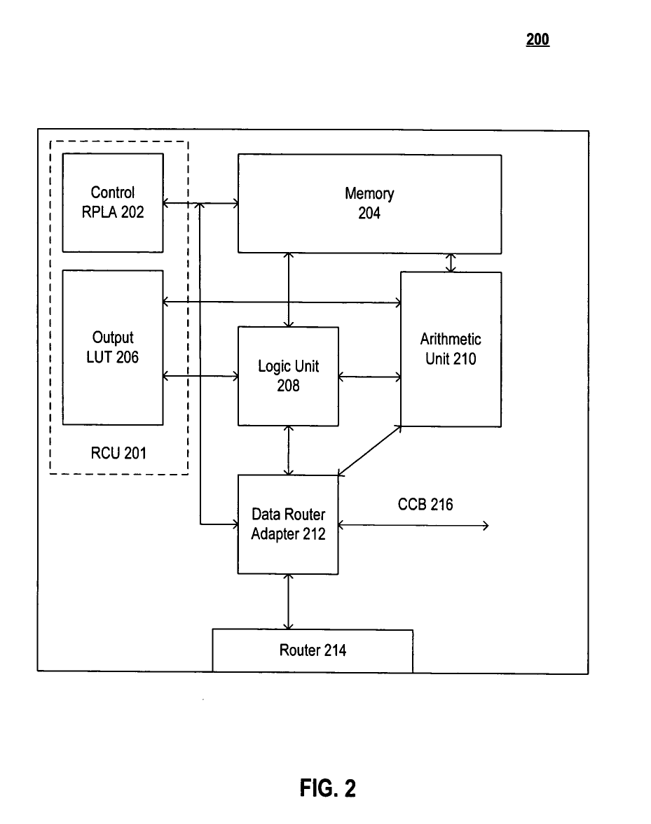

As described elsewhere herein, the PE may include one or more MCAs, where the MCA may be a FMCA as described below with respect to FIG. 2. The present invention is not limited to FMCA or implementations which employ MCAs, as the one or ordinary skill in the art would recognize the reconfigurable control logic employed by the embodiments described herein as providing the requisite capabilities to make use of the programming method, system and tool of the present invention.

With respect to FIG. 2, a diagram of a filter PE 200 which may include one or more MCAs. In embodiments with a single MCA, the PE 200 may also be called a FMCA. A filter PE 200, according to embodiments of the present invention, is illustrated. According to the embodiment shown, the filter PE 200 may include a reconfigurable control unit 201, which may include a control reconfigurable programmable logic array (RPLA) 202 and an output look-up table (LUT) 206. The control RPLA 202 may provide trigger queues and generate control signals for the filter PE 200. The control RPLA 202 may be coupled to the other components of the filter PE 200, and thus able to communicate with one or more of a memory 204, the output LUT 206, a logic unit (LU) 208, an arithmetic unit (AU or arithmetic logic unit (ALU)) 210, and a data router adapter (DRA) 212. The DRA 212 may be coupled to a router 214, either internal or external to the PE 200.

The one or more memory 204 may store control and routing information. In some embodiments of the present invention, the memory 204 may be configured with multiple ports for different functions, such as, but not limited to, storing and retrieving data and coefficient information. The output LUT 206 may receive operation numbers from the control RPLA 202. The output LUT 206 may look-up function and control signals to be forwarded to the other components of the PE 200.

The LU 208 may perform various functions, such as, but not limited to, trigger logic functions, pre-adder controls, dispreader code generation, and communicate with the AU 210 to operate in parallel with it. In some embodiments, the AU 210 may have internal parallelism. The AU 210 may perform various arithmetic functions, such as, but not limited to, adding, multiplying, and other accumulation functions. The AU 210 may also, according to some embodiments, perform enhanced FFT, dispreading, filtering, and other operations. In one embodiment, the data paths of the AU 210 may be reconfigured to perform these and additional functions or operations as may be required by other or new protocols.

The DRA 212 may be coupled one or more of the other components, as well as a command and configuration bus (CCB) 216. The CCB 216 may be coupled to a command and configuration mesh (CCM) (not shown). The DRA 212 may be coupled to a router to one or more networks of other PEs or other components.

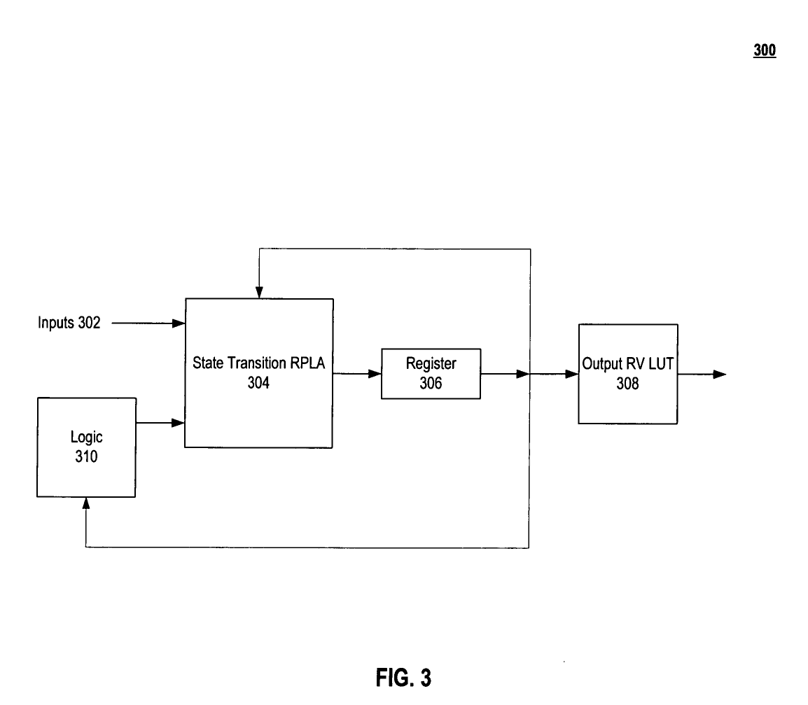

With regard to FIG. 3, a diagram of a control unit (CU) 300 for the PE, according to embodiments of the present invention, is shown. CU 300 may include inputs 302 feeding into a state transition reconfigurable logic array (RPLA) 304. The RPLA 304 may also receive input from a logic 310. According to embodiments of the present invention, the RPLA 304 may be coupled to a register 306. The register 306 may feed into an output RV look-up table (LUT) 308, as well back into the RPLA 304 and logic 310, as illustrated.

In embodiments of the present invention, the control unit 300 may be implemented within the control RPLA 202 of the processing element 200, as one of ordinary skill in the art(s) would recognize based at least on the teachings provided herein.

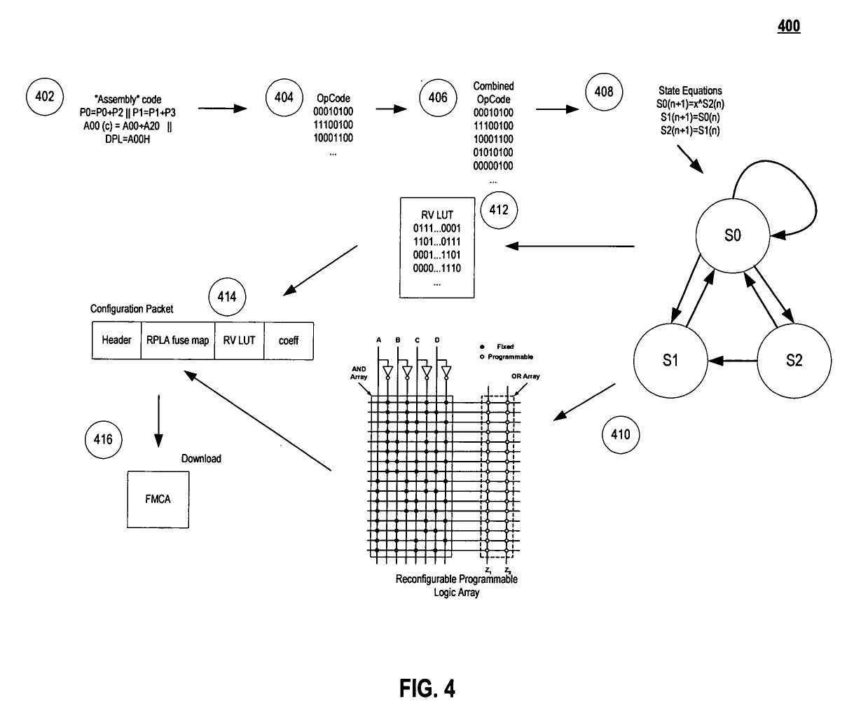

FIG. 4 illustrates a programming output example for the PE, according to embodiments of the present invention. The outputs of FIG. 4 are described with respect to embodiments of the programming tool 100 of the present invention, the instruction converter 104 may receive a function in assembly code 402 and parse the function into a parsed group selected from a group including an instruction, data, a constant, a coefficient, and a factor. Once the function may be parsed, the instruction converter 104 may convert one or more of the parsed group into one or more operation codes 404. An operation code may be a numeric representation for each of the parsed group.

A processing element combiner 108 may determine the one or more operation codes to be executed on a processing element and combine the one or more operation codes into a file 406. The combiner 108 may be coupled to a state equation generator 112, a reconfigurator vector (RV) look-up table generator 116, and a configuration packet generator 118.

The state equation generator 112 may convert the file into a state machine representation 408. The instruction converter 112 may assign an operation number as an output of a state of the one or more operation codes, determine a next state for each of the one or more operation codes, wherein the next state may be a function of the state and an input signal, associate the operation number with a reconfigurator vector, and generate a state equation for each state.

According to embodiments of the present invention, a reconfigurable logic array configurator 114 may map the state equations to a control unit reconfigurable logic array 410 by creating a fuse map for the control unit reconfigurable logic array. The configurator 114 may forward, at least the fuse map to a configuration packet generator 118.

The reconfigurator vector look-up table 116 may produce an output reconfigurator vector look-up table 412, where the reconfigurator vector look-up table converts the operation number to a reconfigurator vector. According to embodiments of the present invention, at least the reconfigurator vector may be forwarded to the configuration packet generator 118.

In embodiments of the present invention, the configuration packet generator 118 may create a configuration packet 414 used to convey the programming information to the PE, that may be, according to an exemplary embodiment, FMCA 416. The configuration packet may include at least one of the fuse map, the output reconfigurator vector look-up table, the parsed group, one or more fuse maps for other control unit reconfigurable logic arrays, routing information, one or more processing element addresses, or packet sizes.

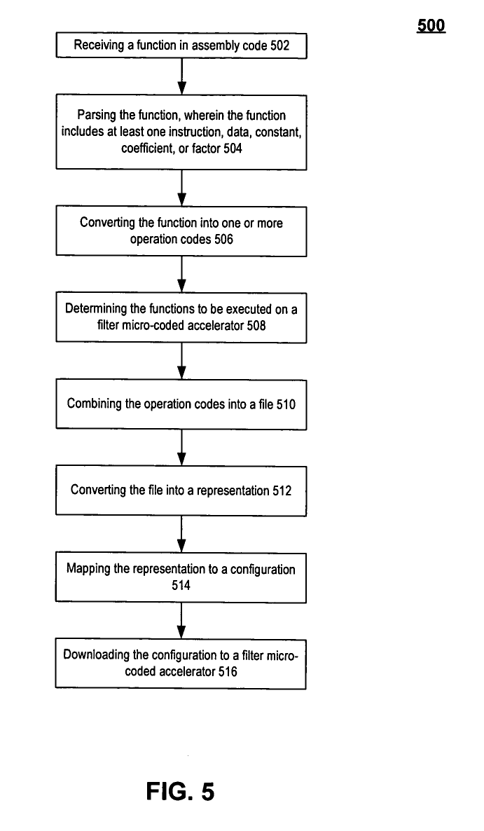

With regard to FIG. 5, a flowchart of the programming operations, according to an embodiment of the present invention, is illustrated. According to embodiments of the present invention, the methods of the tool 100 may include one or more of the following operations performed substantially, but not strictly in the order presented, as one of ordinary skill in the relevant arts would recognize based on the teachings herein.

The process begins at block 502, when the tool 100 may receive a function in assembly code and proceeds immediately to block 504.

In block 504, the tool 100 may parse the function into a parsed group selected from a group including an instruction, data, a constant, a coefficient, and a factor. The process then proceeds to block 506. In block 506, the tool 100 may convert one or more of the parsed group into one or more operation codes, wherein an operation code may be a numeric representation for each of the parsed group.

The process proceeds to block 508, where the tool may determine the one or more operation codes to be executed on a processing element and then proceed to block 510, where it may combine one or more operation codes into a file. The process then proceeds to block 512.

At block 512, the tool 100 may convert the file into a state machine representation. According to embodiments of the present invention, the tool 100 may further assign each of the one or more operation codes a state, wherein an output of the state may be an operation number, determine a next state for each of the one or more operation codes, wherein the next state may be a function of the state and an input signal, associate the operation number with a reconfigurator vector, and generate a state equation for each state.

The process then proceeds to block 514, where the tool 100 may map the state equations to a control unit reconfigurable logic array. In embodiments of the present invention, the tool 100 may program a fuse map for the control unit reconfigurable logic array.

In embodiments of the present invention, the process may produce an output reconfigurator vector look-up table, wherein the reconfigurator vector look-up table converts the operation number to a reconfigurator vector. At block 516, the tool 100 may create a configuration packet used to download and program the processing element.

The configuration packet may include at least one of the fuse map, the output reconfigurator vector look-up table, the parsed group, one or more fuse maps for other control unit reconfigurable logic arrays, routing information, one or more processing element addresses, or packet sizes.

According to the operating environments discussed below, the process of the present invention, according to the embodiments described above, may be implemented in an apparatus designed to perform these operations. Such an apparatus may include software on a personal computer (PC) designed to perform the process described above on the processing element described above.

The present invention (i.e., the programming tool, components of the processing element, etc.) may be implemented using hardware, software or a combination thereof and may be implemented in one or more computing systems or other processing systems. In fact, in one embodiment, the invention may comprise one or more computer or computing systems capable of carrying out the functionality described herein.

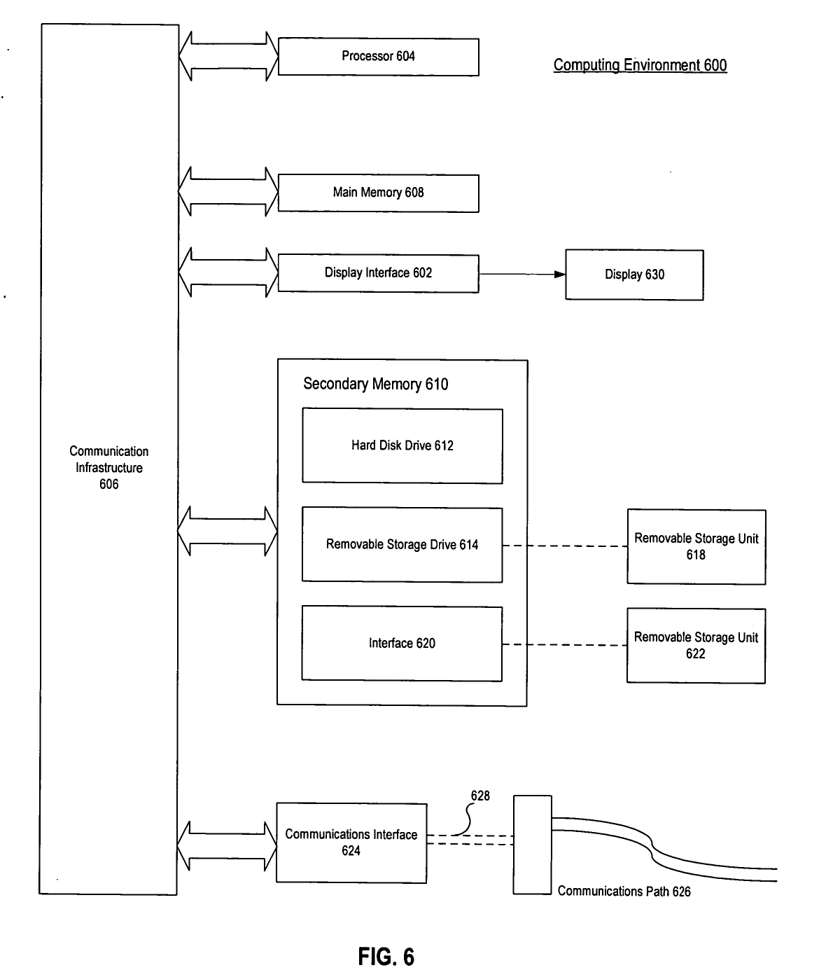

Such computer systems may be implemented in a personal computer (PC), personal digital assistant (PDA), cellular or mobile telephone, pager, kiosk, or some combination of these devices. An example of a computing environment 600 is shown in FIG. 6. The computing environment 600 may include one or more processors, such as processor 604. The processor 604 may be coupled to a communication infrastructure 606 (e.g., a communications bus, cross over bar, or network). Various software embodiments are described in terms of this exemplary computer system. After reading this description, it will become apparent to a person skilled in the relevant art(s) how to implement the invention using other computer systems and/or computer architectures.

Computing environment 600 may include a display interface 602 that may forward graphics, text, and other data from the communication infrastructure 606 (or from a frame buffer not shown) for display on the display unit 630.

Computing environment 600 may also include a main memory 608, preferably random access memory (RAM), and may also include a secondary memory 610. The secondary memory 610 may include, for example, a hard disk drive 612 and/or a removable storage drive 614, representing a floppy disk drive, a magnetic tape drive, an optical disk drive, etc, but which is not limited thereto. The removable storage drive 614 may read from and/or write to a removable storage unit 618 in a well known manner. Removable storage unit 618, may represent a floppy disk, magnetic tape, optical disk, etc. which may be read by and written to by removable storage drive 614. As will be appreciated, the removable storage unit 618 may include a computer usable storage medium having stored therein computer software and/or data.

In alternative embodiments, secondary memory 610 may include other similar means for allowing computer programs or other instructions to be loaded into computing environment 600. Such means may include, for example, a removable storage unit 622 and an interface 620. Examples of such may include, but are not limited to, a program cartridge and cartridge interface (such as that found in video game devices), a removable memory chip (such as an EPROM, or PROM) and associated socket, and/or other removable storage units 622 and interfaces 620 that may allow software and data to be transferred from the removable storage unit 622 to computing environment 600.

Computing environment 600 may also include a communications interface 624. Communications interface 624 may allow software and data to be transferred between computing environment 600 and external devices. Examples of communications interface 624 may include, but are not limited to, a modem, a network interface (such as an Ethernet card), a communications port, a PCMCIA slot and card, and/or a transceiver (or receiver/transmitter combination) configured to operate according to one or more protocols, etc. Software and data transferred via communications interface 624 are in the form of signals 628 which may be, for example, electronic, electromagnetic, optical or other signals capable of being received by communications interface 624. These signals 628 may be provided to communications interface 624 via a communications path (i.e., channel) 626. This channel 626 may carry signals 628 and may be implemented using wire or cable, fiber optics, a phone line, a cellular phone link, an RF link and/or other communications channels.

In this document, the terms “computer program medium” and “computer usable medium” are used to generally refer to media such as, but not limited to, removable storage drive 614, a hard disk installed in hard disk drive 612, and signals 628. These computer program media are means for providing software to computing environment 600.

Computer programs (also called computer control logic) may be stored in main memory 608 and/or secondary memory 610. Computer programs may also be received via communications interface 624. Such computer programs, when executed, enable the computing environment 600 to perform the features of the present invention as discussed herein. In particular, the computer programs, when executed, may enable the processor 604 to perform the present invention in accordance with the above-described embodiments. Accordingly, such computer programs represent controllers of the computing environment 600.

In an embodiment where the invention may be implemented using software, the software may be stored in a computer program product and loaded into computing environment 600 using, for example, removable storage drive 614, hard drive 612 or communications interface 624. The control logic (software), when executed by the processor 604, causes the processor 604 to perform the functions of the invention as described herein.

In another embodiment, aspects of the computing environment, which receive the signal, prior to or after the PHY layer processing of the present invention is implemented primarily in hardware using, for example, hardware components such as MCAs, digital signal processors (DSP), or application specific integrated circuits (ASICs). Implementation of the hardware state machine so as to perform the functions described herein will be apparent to persons skilled in the relevant art(s). As discussed above, the invention may be implemented using any combination of hardware, firmware and software.

While various embodiments of the invention have been described above, it should be understood that they have been presented by way of example, and not limitation. It will be apparent to persons skilled in the relevant art that various changes in form and detail may be made therein without departing from the spirit and scope of the invention. This may be especially true in light of technology and terms within the relevant art(s) that may be later developed. Thus the invention should not be limited by any of the above described exemplary embodiments, but should be defined only in accordance with the following claims and their equivalents.

1. A method, comprising:
receiving a function in assembly code;
parsing said function into a parsed group selected from a group consisting of an instruction, data, a constant, a coefficient, and a factor;
converting one or more of said parsed group into one or more operation codes, wherein an operation code is a numeric representation for each of said parsed group;
determining said one or more operation codes to be executed on a processing element;
combining said one or more operation codes into a file; and
converting said file into a state machine representation.

2. The method of claim 1, said converting said file into a state machine representation further comprises:
assigning each of said one or more operation codes a state, wherein an output of said state is an operation number;
determining a next state for each of said one or more operation codes, wherein said next state is a function of said state and an input signal;
associating said operation number with a reconfigurator vector; and
generating a state equation for each state.

3. The method of claim 2, further comprising:
mapping said state equations to a control unit reconfigurable logic array; and
programming a fuse map for said control unit reconfigurable logic array.

4. The method of claim 3, further comprising:
producing an output reconfigurator vector look-up table, wherein said reconfigurator vector look-up table converts said operation number to a reconfigurator vector.

5. The method of claim 4, further comprising:
creating a configuration packet;
downloading said configuration packet to said processing element; and
programming said processing element with said configuration packet.

6. The method of claim 5, wherein said configuration packet includes data selected from a group consisting of said fuse map, said output reconfigurator vector look-up table, said parsed group, one or more fuse maps for other control unit reconfigurable logic arrays, routing information, one or more processing element addresses, and packet sizes.

7. The method of claim 1, wherein said processing element includes as least one micro-coded accelerator.

8. A machine-readable medium that provides instructions, which when executed by a computing platform, cause said computing platform to perform operations comprising a method of:
receiving a function in assembly code;
parsing said function into a parsed group selected from a group consisting of an instruction, data, a constant, a coefficient, and a factor;
converting one or more of said parsed group into one or more operation codes, wherein an operation code is a numeric representation for each of said parsed group;
determining said one or more operation codes to be executed on a processing element;
combining said one or more operation codes into a file; and
converting said file into a state machine representation.

9. The machine-readable medium of claim 8, providing further instructions, which when executed by a computing platform, cause the computing platform to perform a further operation of:
assigning each of said one or more operation codes a state, wherein an output of said state is an operation number;
determining a next state for each of said one or more operation codes, wherein said next state is a function of said state and an input signal;
associating said operation number with a reconfigurator vector; and
generating a state equation for each state.

10. The machine-readable medium of claim 9, providing further instructions, which when executed by a computing platform, cause the computing platform to perform a further operation of:
mapping said state equations to a control unit reconfigurable logic array; and
programming a fuse map for said control unit reconfigurable logic array.

11. The machine-readable medium of claim 10, providing further instructions, which when executed by a computing platform, cause the computing platform to perform a further operation of:
producing an output reconfigurator vector look-up table, wherein said reconfigurator vector look-up table converts said operation number to a reconfigurator vector.

12. The machine-readable medium of claim 11, providing further instructions, which when executed by a computing platform, cause the computing platform to perform a further operation of:
creating a configuration packet;
downloading said configuration packet to said processing element; and
programming said processing element with said configuration packet.

13. The machine-readable medium of claim 12, wherein said configuration packet includes data selected from a group consisting of said fuse map, said output reconfigurator vector look-up table, said parsed group, one or more fuse maps for other control unit reconfigurable logic arrays, routing information, one or more processing element addresses, and packet sizes.

14. The machine-readable medium of claim 8, wherein said processing element includes as least one micro-coded accelerator.

15. A system, comprising:
an instruction converter to receive a function in assembly code and to parse said function into a parsed group selected from the group consisting of an instruction, data, a constant, a coefficient, and a factor;
said instruction converter to convert said parsed group into one or more operation codes, wherein each of said operation codes is a numeric representation for each selection of said parsed group;
a processing element combiner to determine said one or more operation codes to be executed on a processing element and combine said one or more operation codes into a file; and
a state equation generator to convert said file into a state machine representation.

16. The system of claim 15, further comprising:
said instruction converter (i) to assign an operation number as an output of a state of said one or more operation codes, (ii) to determine a next state for each of said one or more operation codes, wherein said next state is a function of said state and an input signal, (iii) to associate said operation number with a reconfigurator vector, and (iv) to generate a state equation for each state.

17. The system of claim 16, further comprising:
a reconfigurable logic array configurator to map said state equations to a control unit reconfigurable logic array, and program a fuse map for said control unit reconfigurable logic array.

18. The system of claim 17, further comprising:
a reconfigurator vector look-up table to produce an output reconfigurator vector look-up table, said reconfigurator vector look-up table to convert said operation number to a reconfigurator vector.

19. The system of claim 18, further comprising:
a configuration packet generator to create a configuration packet and to program said processing element with said configuration packet.

20. The system of claim 19, wherein said configuration packet includes data selected from a group consisting of said fuse map, said output reconfigurator vector look-up table, said parsed group, one or more fuse maps for other control unit reconfigurable logic arrays, routing information, one or more processing element addresses, and packet sizes.

21. The system of claim 15, wherein said processing element includes as least one micro-coded accelerator.
